(19) United States
(12) Patent Application Publication
(10) Pub. No.: US 2024/0351018 A1
(43) Pub. Date: Oct. 24, 2024

(54) AUTOMATED OPTICAL MICROPIPETTE ELECTRODE GUIDANCE SYSTEM AND METHOD

(71) Applicants: Ethan Marschall, Queen Creek, AZ (US); Barbara Smith, Tempe, AZ (US)

(72) Inventors: Ethan Marschall, Queen Creek, AZ (US); Barbara Smith, Tempe, AZ (US)

(73) Assignee: Arizona Board of Regents on behalf of Arizona State University, Scottsdale, AZ (US)

(21) Appl. No.: 18/628,195

(22) Filed: Apr. 5, 2024

Related U.S. Application Data
(60) Provisional application No. 63/494,478, filed on Apr. 6, 2023.

(51) Int. Cl. B01L 3/02; G01N 35/00

(52) U.S. Cl. CPC B01L 3/0237; G01N 35/00584; B01L 2300/0654

(57) ABSTRACT

An optical system, comprises a mirror cage, a light source optically connected to a first side of the mirror cage via a first fiberoptic cable and a first collimator, an energy sensor optically connected to a second side of the mirror cage opposite the first side, a second fiber optic cable optically connected to a third side of the mirror cage via a second collimator, an avalanche photodiode (APD) optically connected to a fourth side of the mirror cage opposite the third side, and a dichroic mirror positioned within the housing at an angle relative to the first side. An automated micropipette electrode guidance system comprises the optical system as above, and a micropipette electrode connected to the optical system via the second fiber optic cable. Related methods are also disclosed.

### CROSS-REFERENCE TO RELATED APPLICATIONS

This application claims priority to U.S. Provisional Patent Application No. 63/494,478, filed on Apr. 6, 2023, incorporated herein by reference in its entirety.

### STATEMENT REGARDING FEDERALLY SPONSORED RESEARCH OR DEVELOPMENT

This invention was made with government support under 1944846 awarded by the National Science Foundation. The government has certain rights in the invention.

### BACKGROUND OF THE INVENTION

Micropipettes have been utilized in many fields ranging from cell manipulation to conductance microscopy and electrophysiology. Current optical micropipettes including electrodes lack simultaneous emission and collection of light. Furthermore, some in vivo procedures currently require incremental steps of repeated measurements and movements to find a target cell which have a low rate of success. Thus, there is a need in the art for improved automated optical micropipette electrode guidance systems and methods.

### SUMMARY OF THE INVENTION

Some embodiments of the invention disclosed herein are set forth below, and any combination of these embodiments (or portions thereof) may be made to define another embodiment.

In one aspect, an optical system comprises a mirror cage, a light source optically connected to a first side of the mirror cage via a first fiberoptic cable and a first collimator, an energy sensor optically connected to a second side of the mirror cage opposite the first side, a second fiber optic cable optically connected to a third side of the mirror cage via a second collimator, an avalanche photodiode (APD) optically connected to a fourth side of the mirror cage opposite the third side, and a dichroic mirror positioned within the housing at an angle relative to the first side.

In one embodiment, the second fiber optic cable comprises a tapered fiber optic cable.

In one embodiment, the dichroic mirror is positioned at an angle of 45 degrees relative to the first side.

In one embodiment, the dichroic mirror is positioned at an angle of 45 degrees relative to each of the first, second, third and fourth sides.

In one embodiment, the dichroic mirror is planar, concave or convex.

In one embodiment, the light source comprises a laser.

In one embodiment, the laser is configured to provide light in the range of 400 to 600 nm.

In one embodiment, the at least one filter is configured to focus light on the APD.

In one embodiment, the at least one filter comprises five filters.

In one embodiment, the dichroic mirror is configured to reflect light in the range of 400 to 600 nm.

In one embodiment, the system further comprises a plurality of gaskets positioned between the first collimator and the mirror cage, the second collimator and the mirror cage, and the APD and the mirror cage.

In one embodiment, the first and second fiber optic cables comprise FC/PC fiber optic cables.

In one embodiment, the system further comprises a neutral density filter position between the energy sensor and the mirror cube.

In one embodiment, the APD is directly connected to the fourth side of the mirror cage via a portion housing at least one filter.

In one embodiment, the APD is indirectly connected to the fourth side of the mirror cage via a portion housing at least one filter and a third fiberoptic cable.

In another aspect, an automated micropipette electrode guidance system comprises the optical system as described above, and a micropipette electrode connected to the optical system via the second fiber optic cable.

In one embodiment, the system is configured for simultaneous emission and collection of light from the micropipette electrode.

In another aspect, an automated micropipette electrode guidance method comprises providing the optical system as described above, providing a micropipette electrode connected to the optical system via the second fiber optic cable, supplying a first portion of light to the micropipette electrode via the light source and optical system, measuring a second portion of light on the energy sensor, and measuring collected light from the micropipette electrode with the APD.

In one embodiment, the step of supplying a first portion of light to the micropipette electrode comprises supplying light via the light source, the first fiber optic cable, the first collimator, the dichroic mirror, the second collimator, and the second fiber optic cable to the micropipette electrode.

In one embodiment, the step of measuring a second portion of light comprises measuring light supplied by the light source that was transmitted through the dichroic mirror.

In one embodiment, the step of measuring collected light from the micropipette electrode with the APD comprises measuring light received from the micropipette electrode via the second fiber optic cable, the second collimator, the dichroic mirror, and the at least one filter.

In one embodiment, the supplied light is in the range of 400 to 600 nm and is supplied by a laser.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 depicts an exemplary automated optical micropipette electrode guidance system in accordance with some embodiments.

FIGS. 2A through 2F depict perspective views with exemplary component details of an exemplary experimental automated optical micropipette electrode guidance system in accordance with some embodiments.

FIG. 3 depicts a cutaway view of a portion of an exemplary experimental automated optical micropipette electrode guidance system in accordance with some embodiments.

FIG. 4 depicts a system level view of components of an exemplary automated optical micropipette electrode guidance system in accordance with some embodiments.

FIGS. 5A and 5B depict perspective views with exemplary component details of another exemplary experimental automated optical micropipette electrode guidance system in accordance with some embodiments.

FIGS. 6A and 6B depict cutaway views of portions of another exemplary experimental automated optical micropipette electrode guidance system in accordance with some embodiments.

FIGS. 7A and 7B depict images of an exemplary experimental avalanche photodiode (APD) and bracket for use in an exemplary experimental automated optical micropipette electrode guidance system in accordance with some embodiments.

FIG. 8 depicts an image of an exemplary experimental automated optical micropipette electrode guidance system in accordance with some embodiments.

FIGS. 9A and 9B depict images of another exemplary experimental automated optical micropipette electrode guidance system in accordance with some embodiments.

FIGS. 10A-10D depict images of another exemplary experimental automated optical micropipette electrode guidance system in accordance with some embodiments.

FIGS. 11A and 11B depict an exemplary APD bracket for use in an exemplary experimental automated optical micropipette electrode guidance system in accordance with some embodiments.

FIG. 12 depicts an exemplary ambient light blocker for use in an exemplary experimental automated optical micropipette electrode guidance system in accordance with some embodiments.

### DETAILED DESCRIPTION OF THE INVENTION

It is to be understood that the figures and descriptions of the present invention have been simplified to illustrate elements that are relevant for a clearer comprehension of the present invention, while eliminating, for the purpose of clarity, many other elements found in systems and methods of automated optical micropipette electrodes. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass the specified value or variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein are automated optical micropipette electrode guidance systems and methods.

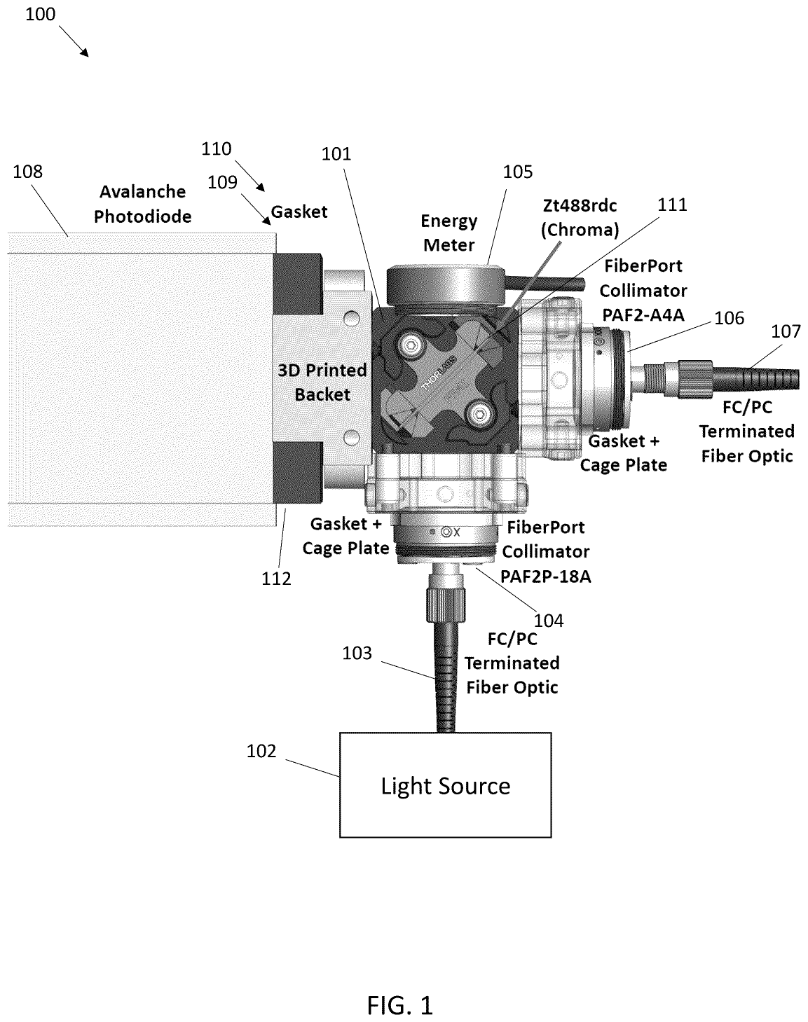

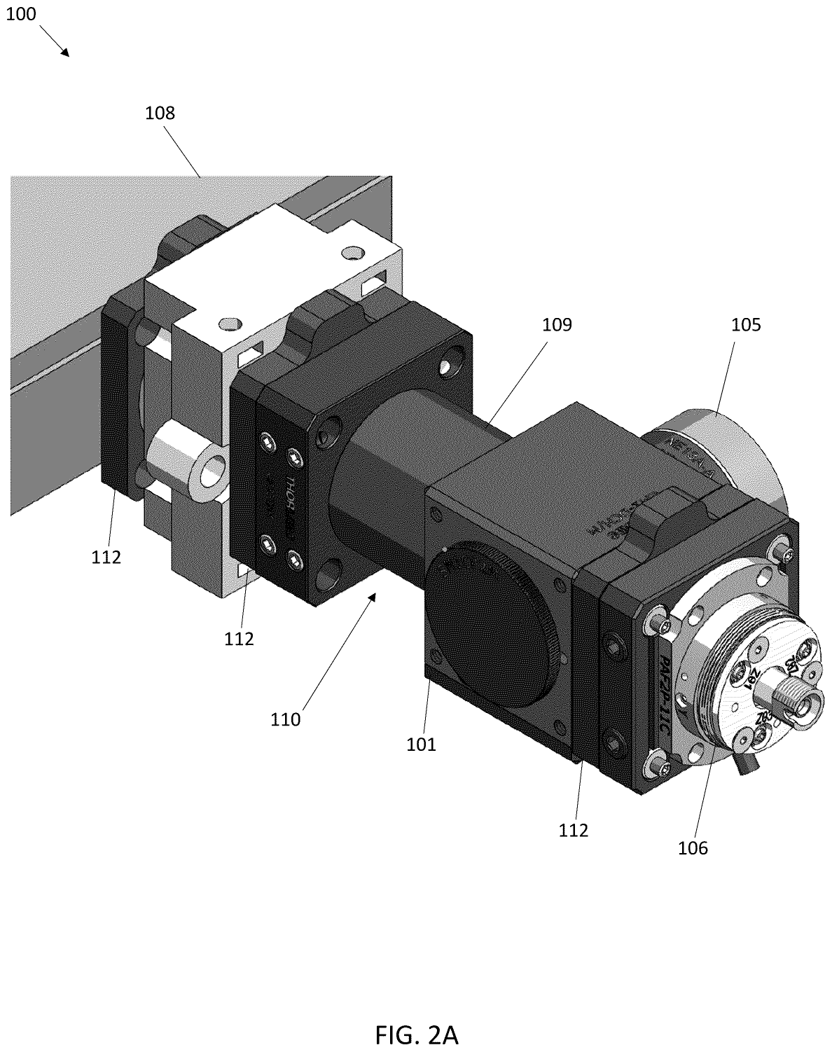

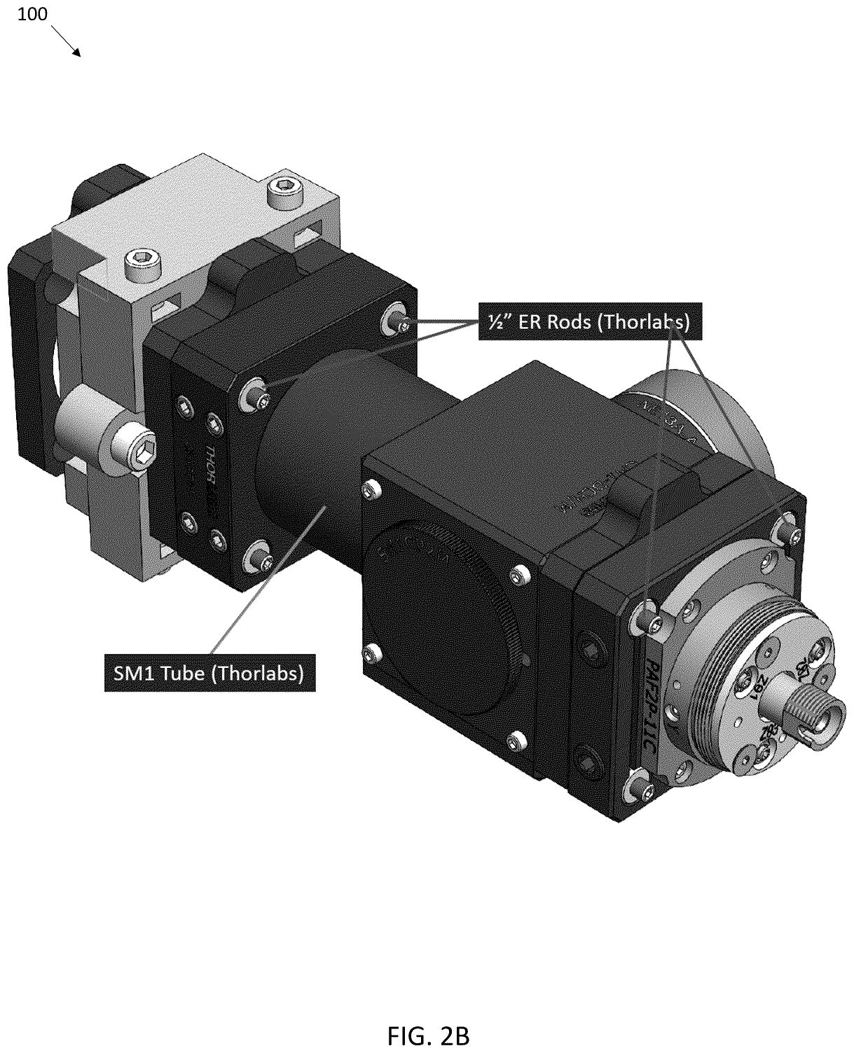

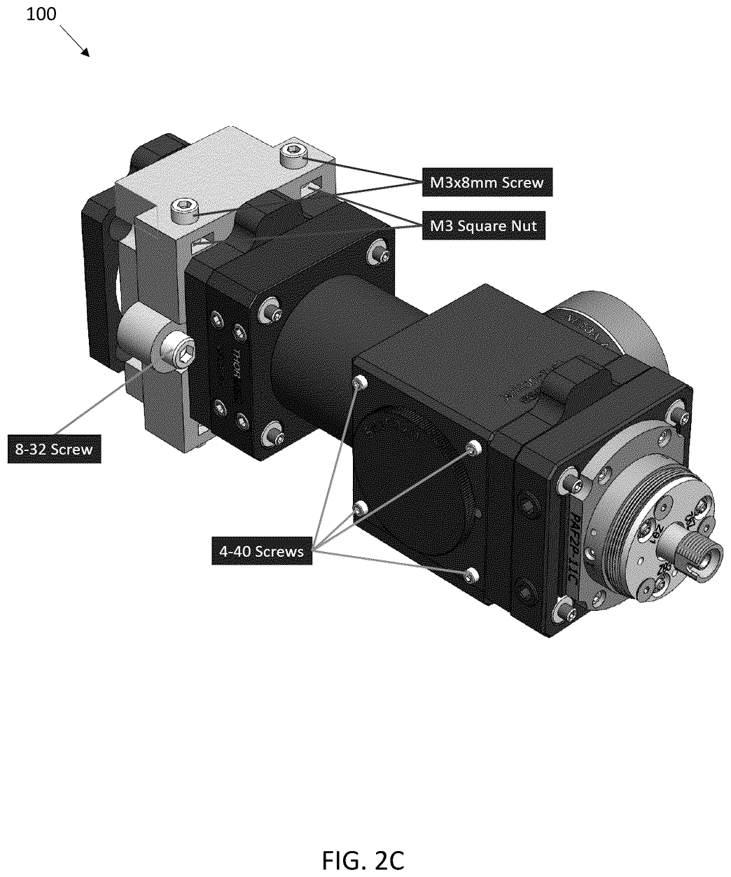

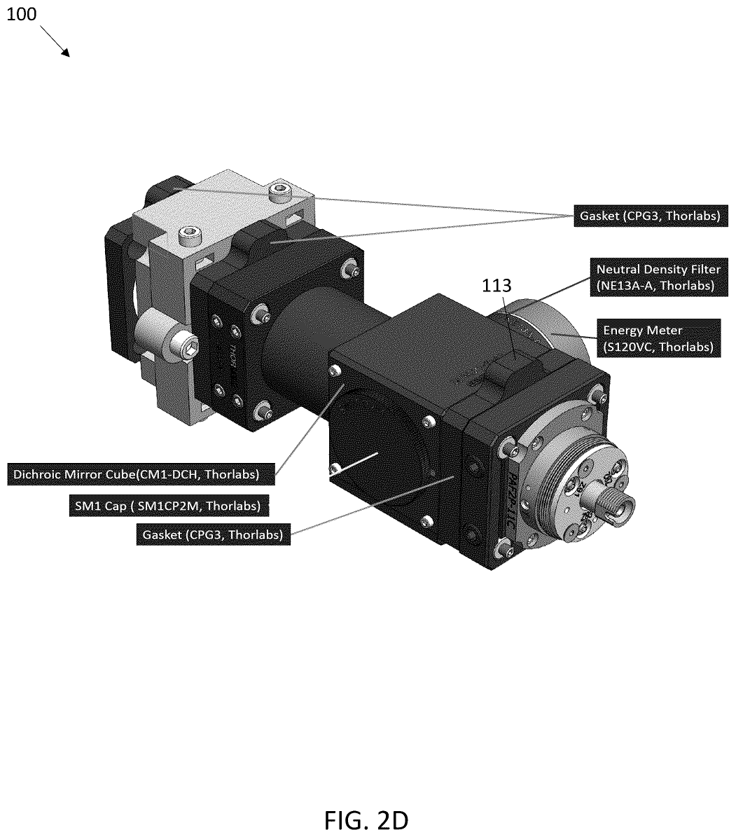

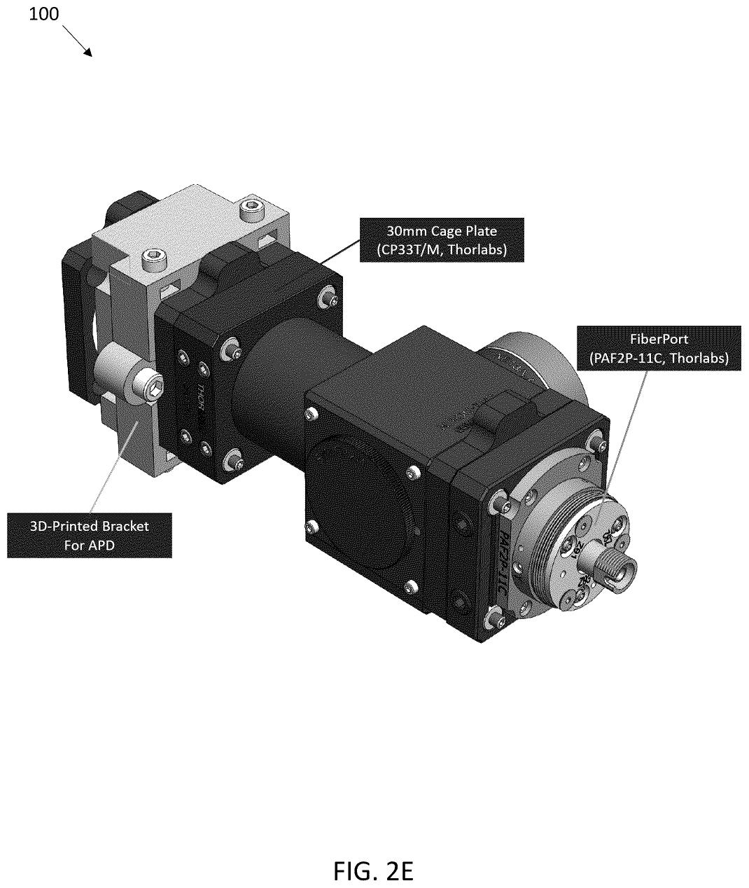

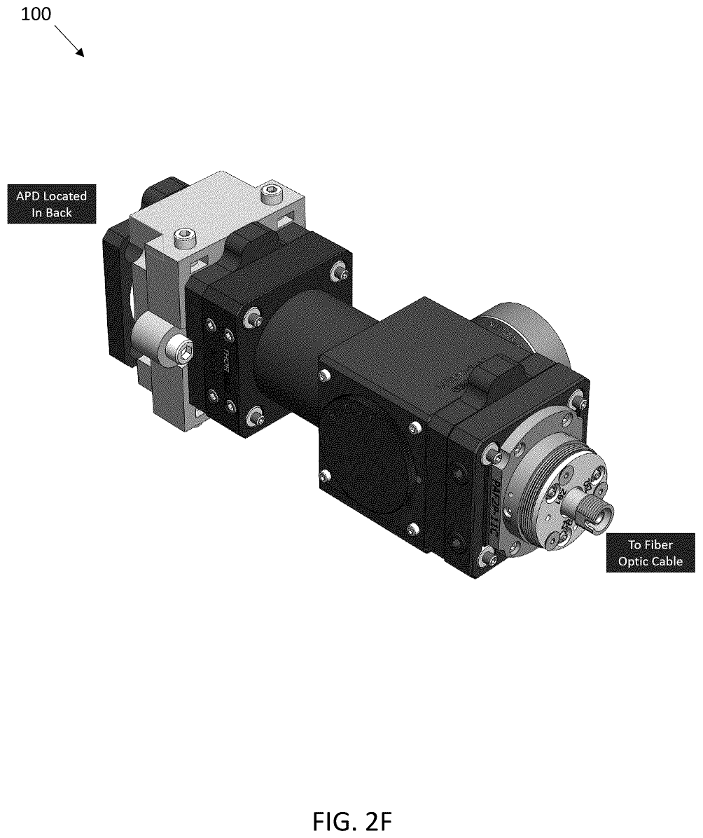

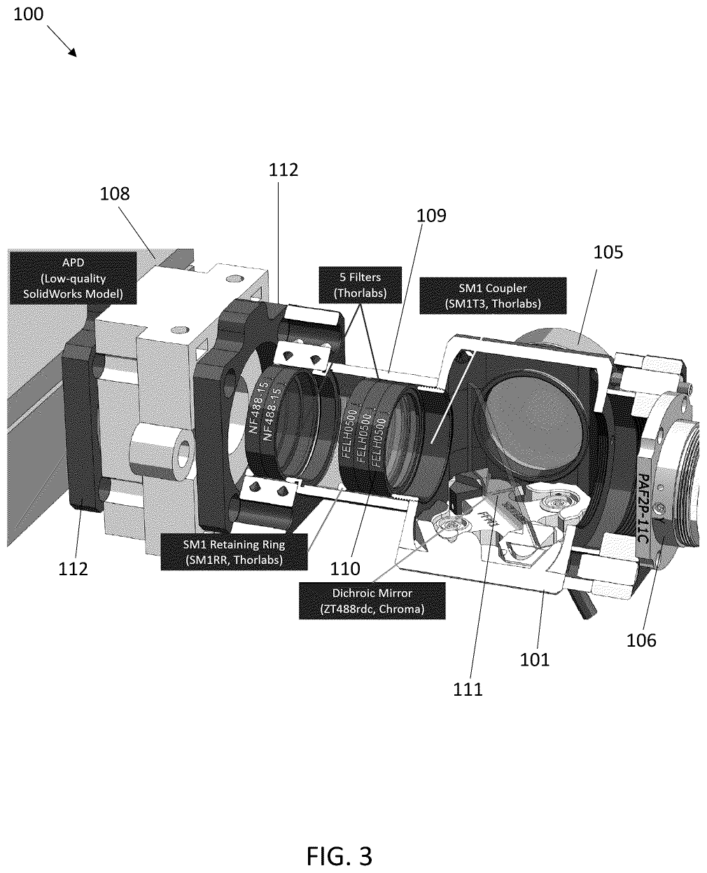

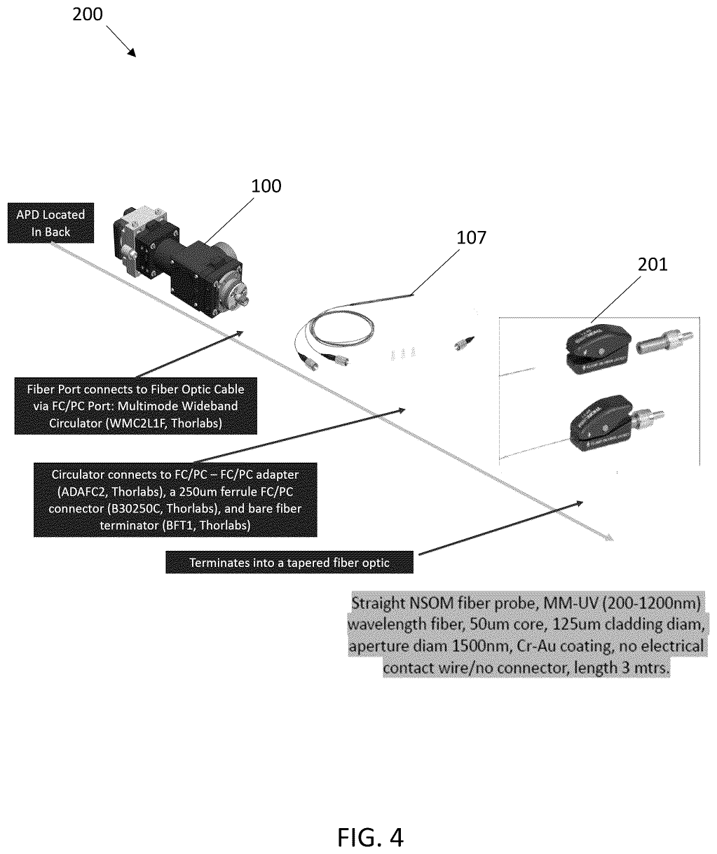

Referring now to FIG. 1, FIGS. 2A-2F, FIG. 3, and FIG. 4 an exemplary automated optical micropipette electrode guidance system 100 is shown. FIG. 1 is a layout of an exemplary optical system 100 of the automated optical micropipette electrode guidance system 200. FIGS. 2A-2F are perspective views of the optical system 100 including exemplary component details. FIG. 3 is a cutaway view of a portion of the optical system 100. FIG. 4 is a system level view of components of the exemplary automated optical micropipette electrode guidance system.

In some embodiments, an optical system 100 includes a mirror cage 101, a light source 102 optically connected to a first side of the mirror cage 101 via a first fiberoptic cable 103 and a first collimator 104. In some embodiments, the optical system 100 further includes an energy sensor 105 (for example, S120Vc, Thorlabs) optically connected to a second side of the mirror cage 101 opposite the first side. In some embodiments, the optical system 100 further includes a second fiber optic cable 107 optically connected to a third side of the mirror cage 101 via a second collimator 106. In some embodiments, the optical system 100 further includes an avalanche photodiode (APD) 108 optically connected to a fourth side of the mirror cage 101 opposite the third side via a filter portion 109 housing at least one filter 110 (for example NF488-15 and/or FELH0500, Thorlabs). In some embodiments, the optical system 100 further includes a dichroic mirror 111 (for example, ZT488rdc, Chroma) positioned within the housing at an angle relative to the first side.

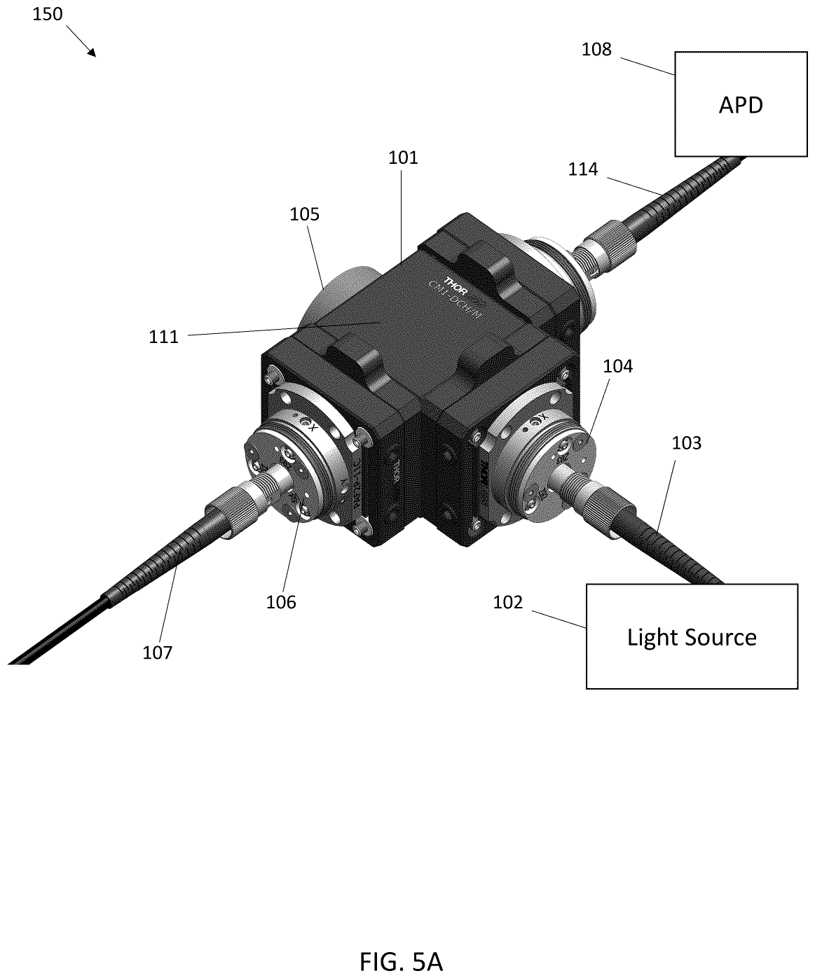

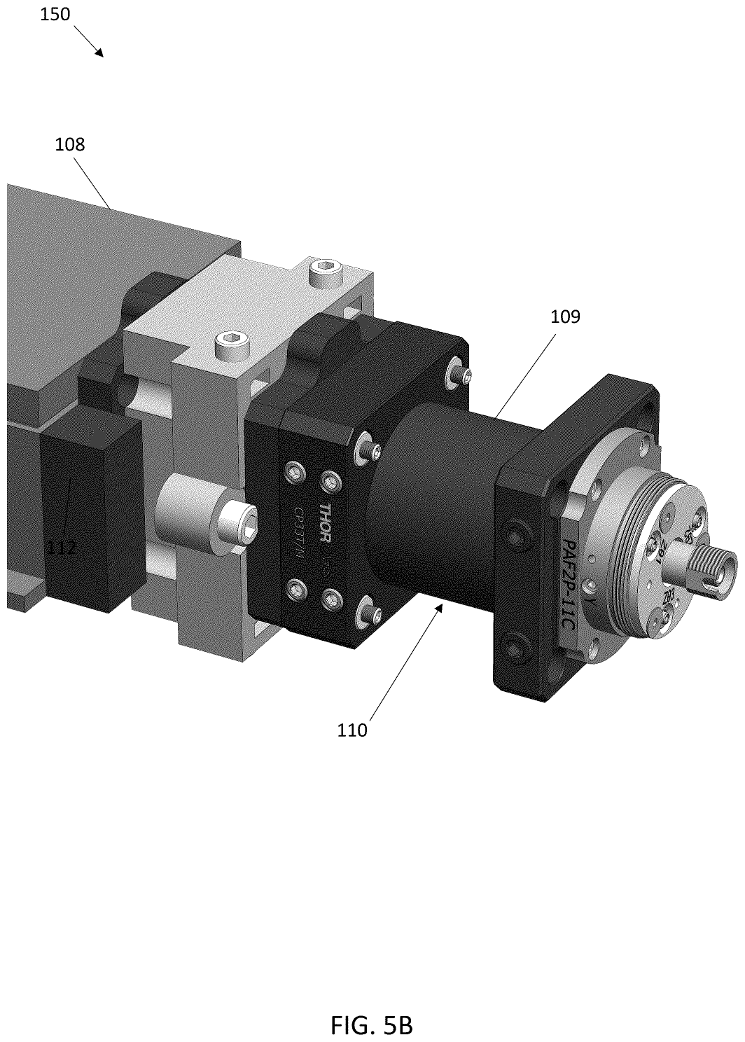

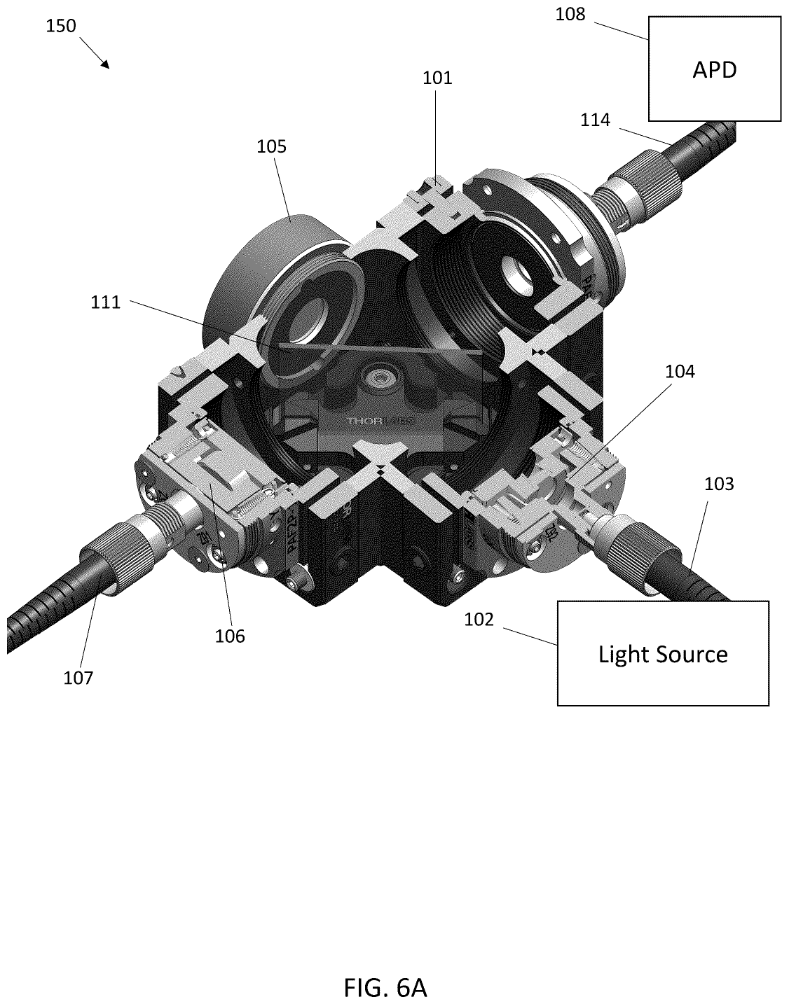

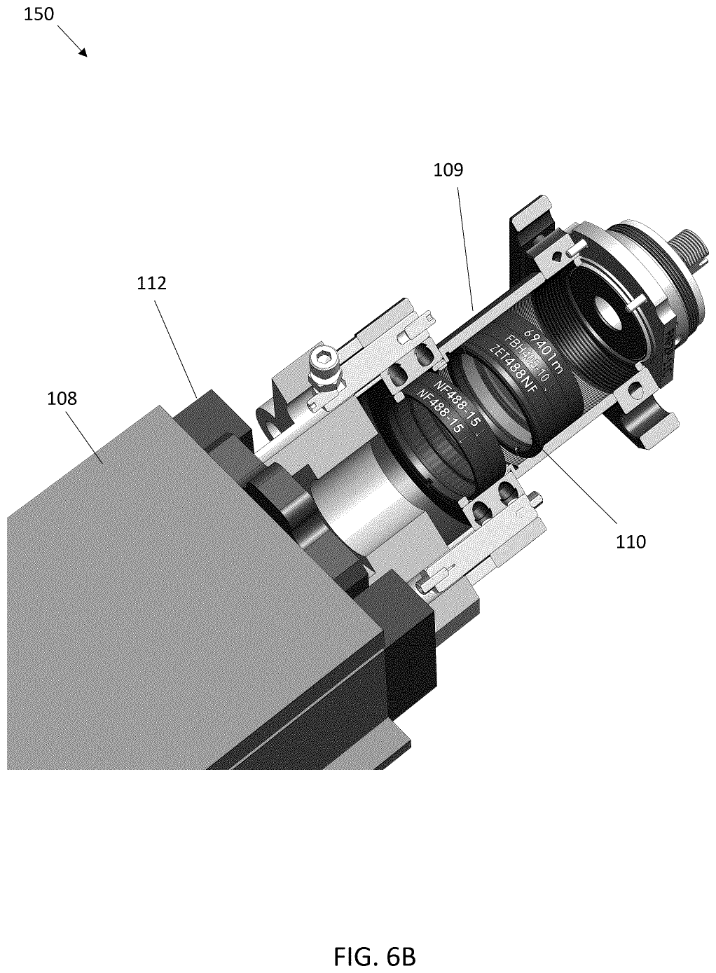

Referring now to FIGS. 5A-5B, and FIGS. 6A-6B another exemplary automated optical micropipette electrode guidance system 150 is shown. FIGS. 5A and 5B depict perspective views with exemplary component details of system 150. FIGS. 6A and 6B depict cutaway views of portions system 150.

In some embodiments, an optical system 150 includes a mirror cage 101, a light source 102 optically connected to a first side of the mirror cage 101 via a first fiberoptic cable 103 and a first collimator 104. In some embodiments, the optical system 150 further includes an energy sensor 105 (for example, S120Vc, Thorlabs) optically connected to a second side of the mirror cage 101 opposite the first side. In some embodiments, the optical system 150 further includes a second fiber optic cable 107 optically connected to a third side of the mirror cage 101 via a second collimator 106. In some embodiments, the optical system 150 further includes an avalanche photodiode (APD) 108 optically connected to a fourth side of the mirror cage 101 opposite the third side via a filter portion 109 housing at least one filter 110 (for example NF488-15 and/or FELH0500, Thorlabs) and a third fiber optic cable 114. In some embodiments, the optical system 150 further includes a dichroic mirror 111 (for example, ZT488rdc, Chroma) positioned within the housing at an angle relative to the first side.

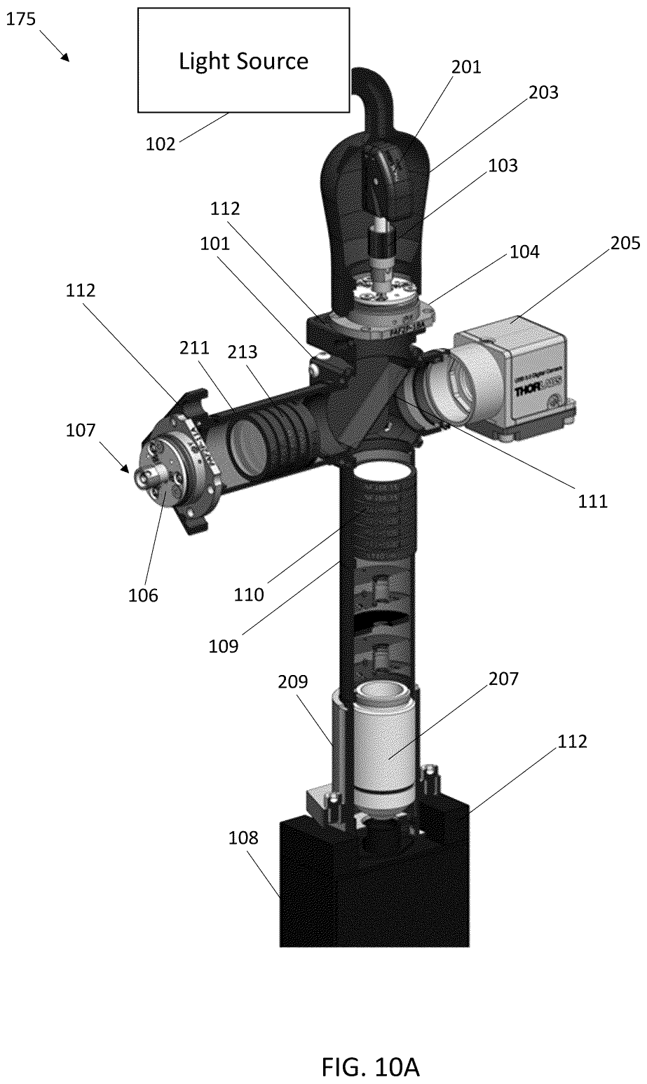

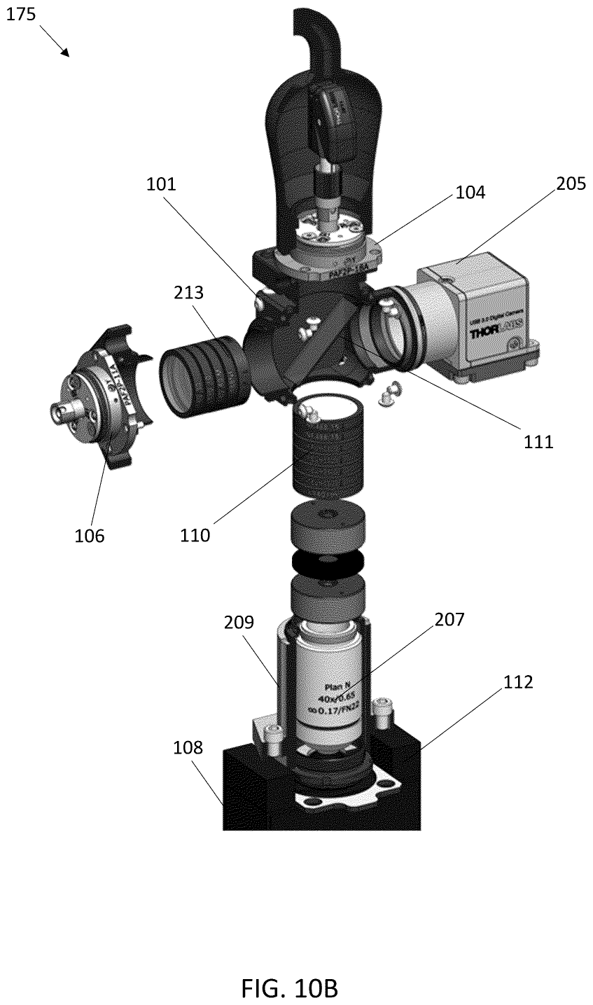

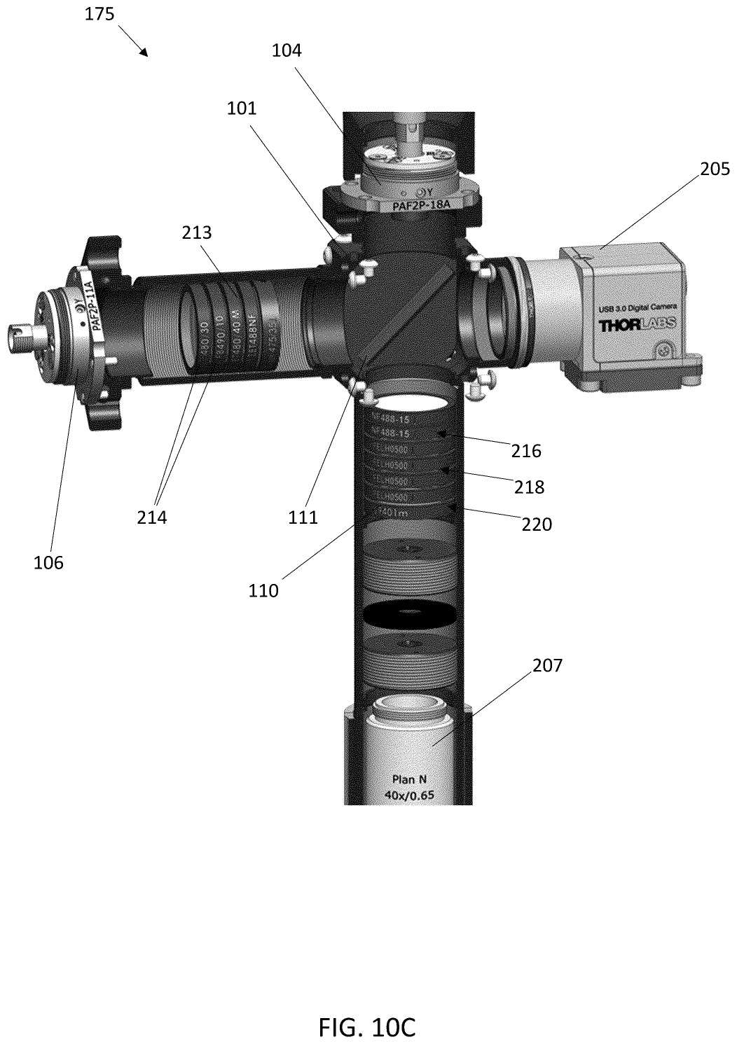

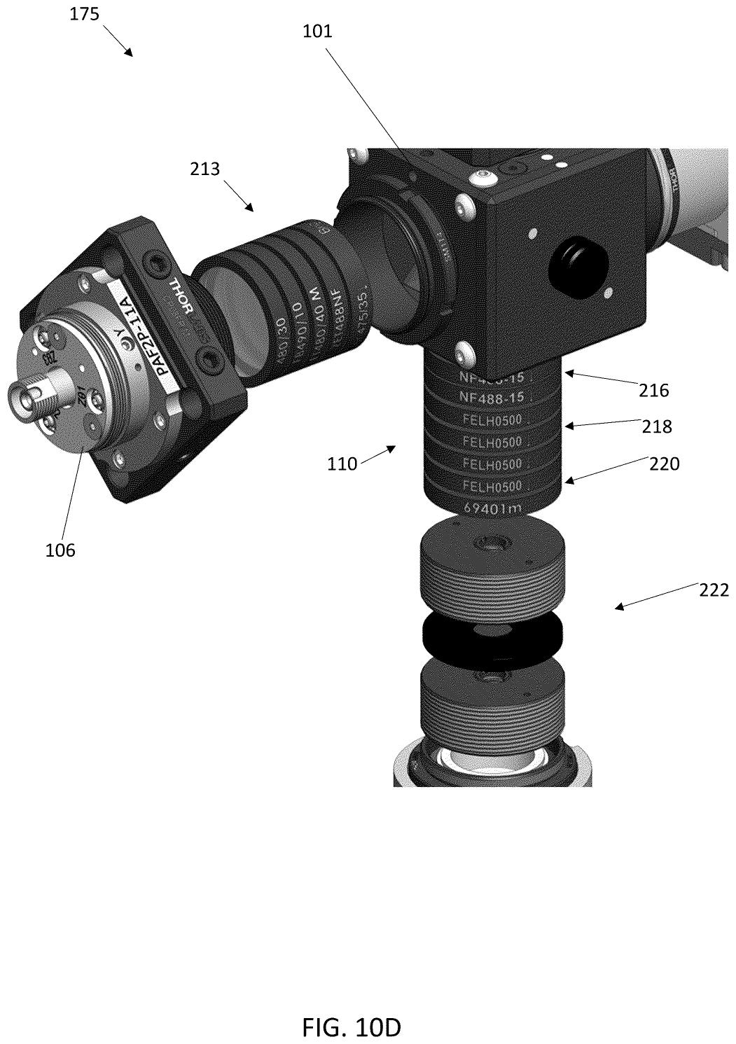

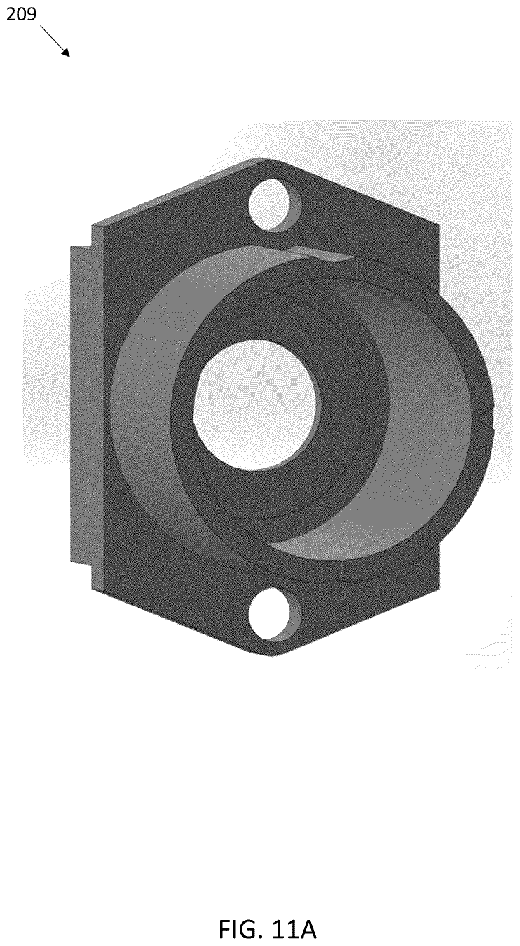

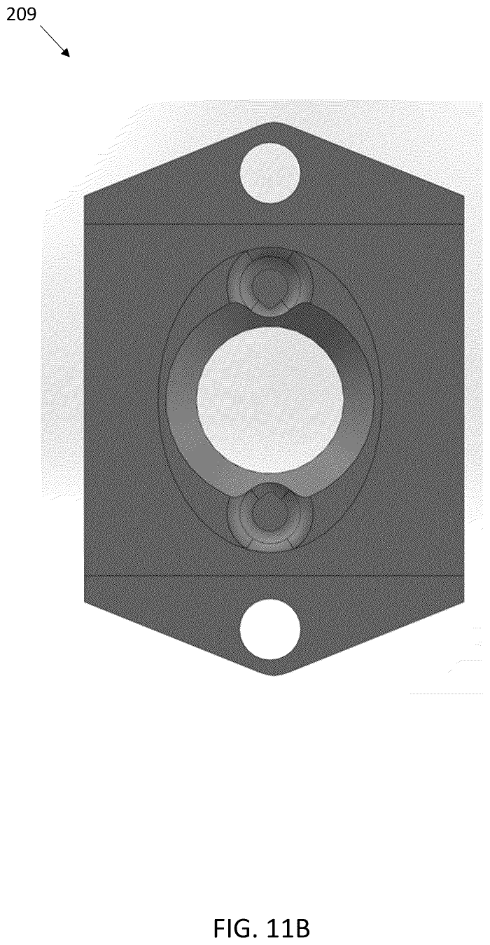

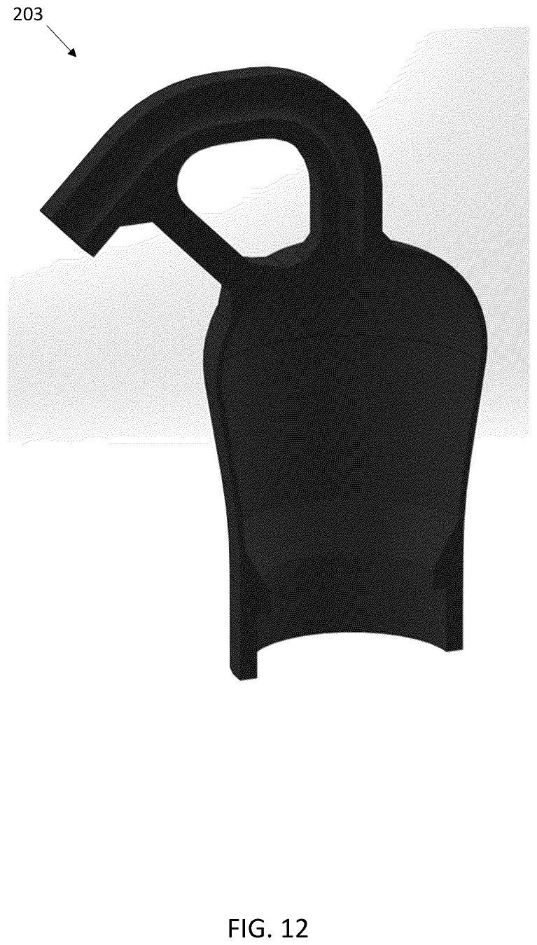

Referring now to FIGS. 10A-10D, 11A, 11B, and 12, another exemplary automated optical micropipette electrode guidance system 175 is shown. FIGS. 10A-10D depict perspective cross-sectional views of exemplary component details of system 175. FIGS. 11A and 11B depict perspective views of an APD mount 209. FIG. 12 depicts an cross-sectional view of an ambient light blocker 203.

In some embodiments, an optical system 175 includes a mirror cage 101, a light source 102 optically connected to a first side of the mirror cage 101 via a first fiberoptic cable 103 and a first collimator 104. In some embodiments, the optical system 175 further includes a micropipette electrode 201 connected to the optical system via the first fiberoptic cable 103. The micropipette electrode 201 may be a fiber optic clamp with a tolerance to sit over lens tubes (for example SM1 lens tubes). The ambient light blocker 203 encases the micropipette electrode 201 and at least a portion of the first fiberoptic cable 103. By encasing the micropipette electrode 201 and at least a portion of the first fiberoptic cable 103, the ambient light blocker 203 prevents ambient light from entering into the lens tube system (for example system 175) and/or the side of the first fiberoptic cable 103. As such, the system 175 and the APD 108 may be operated with lights illuminating a room housing the system 175.

In some embodiments, the optical system 175 further includes the APD 108 optically connected to a second side of the mirror cage 101 opposite the first side via a filter portion 109 housing at least one filter 110 and an objective 207 (for example a 40× objective, 10× objective, and the like) to focus the beam onto the APD 108. The focus of the objective 207 may be determined based on design parameters of the system 175 (for example spot size, focal length, size of objective, threading, and the like). An APD mount 209 may be coupled to the APD 108 via a gasket 112. The APD mount 209 may be a tubular body in which a tube assembly may be positioned within the tubular body via a proximal end opening. The tube assembly may comprise for example at least one filter 110 and an objective 207 housed within the filter portion 109. The entire lens-tube assembly may be slipped out for easy access. The lens-tube assembly may be held in place within the APD mount 209 via gravity and friction. In some embodiments, the APD mount 209 and/or ambient light blocker 203 may be constructed of one or more materials (for example Polylactic acid (PLA), Polytetrafluoroethylene (PTFE), Acrylonitrile Butadiene Styrene (ABS), and other like materials).

The at least one filter 110 includes one or more notch filters 216 (for example NF488-15, Thorlabs), one or more high-pass filters 218 (for example FELH0500, Thorlabs), one or more multi-bandpass filters 220 (for example 69401m, Chroma), and/or one or more spatial filters 222. The one or more notch filters 216 may block out reflected light (for example 488 nm light) from the laser. The one or more notch filters 216 may allow emission light from EGFP fluorescently-labeled neurons. The one or more high-pass filters 218 may block out reflected light (for example 488 nm light) from the laser. The one or more high-pass filters 218 may block out noise from low-wavelength light. The one or more high-pass filters 218 may allow emission light from EGFP fluorescently-labeled neurons. The one or more multi-bandpass filters 220 blocks out some high-wavelength noise, but also blocks out the reflected light (for example 488 nm light) from the laser. The one or more multi-bandpass filters 220 may allow emission light from EGFP fluorescently-labeled neurons. The spatial filter 222 may include two N-BK7 6 mm-diameter, focal length of 10 mm (f=10) plano-convex lenses (LA1116, Thorlabs) disposed on opposite sides of a stainless steel 50 um pinhole (P50K, Thorlabs). In some cases, the spatial filter 222 may use lenses having 12 mm and 25 mm diameters. In some cases, the spatial filter 222 may use lenses having longer focal lengths (for example 20 mm, 50 mm, 100 mm). In some cases, the spatial filter 222 may use alternative lenses having different lens substrates (for example Thorlabs optical substrates, such as, N-BK7, Silica, N-SF11, CaF2, MgF2, and the like). In some cases, the spatial filter 222 may use lenses having no coating, or -A, -AB, -B antireflective coating. In some cases, the spatial filter 222 may use a different pinhole diameter (e.g. 25 um, 75 um, 100 um). In some cases, the spatial filter 222 may use a pinhole having a material different than stainless steel.

In some embodiments, the optical system 175 further includes a second fiber optic cable 107 optically connected to a third side of the mirror cage 101 via a second collimator 106. In some embodiments, the collimator 106 is connected to the mirror cage 101 via a bracket, a cage plate (for example, CP33T/M, Thorlabs), a 3D printed bracket, and/or the filter portion 211. In some embodiments, the filter portion 211 comprises a tube (for example, SM1 Tube, Thorlabs). In some embodiments, the filter portion 211 is connected to the mirror cage 101 via coupler (for example, SM1T3, Thorlabs). In some embodiments, the filter portion 211 includes a filter retaining ring (for example, SM1RR, Thorlabs). The filter portion 211 includes at least one clean-up filter 213. For example, the filter portion 211 includes five filters (for example filters 480/30, and filter FB490/10). The at least one filter 213 cleans up emission from the laser (for example, the 488 nm laser) to ensure that the incoming light has a narrow wavelength window (for example, a very narrow wavelength window).

In some embodiments, the optical system 175 further includes a camera 205 (for example a DCC3240C camera) optically connected to a fourth side of the mirror cage 101 opposite the third side of the mirror cage 101. The camera 205 may image a percentage (for example a small percentage) of incoming 488 nm laser light to align the laser. In some cases, the laser may be also aligned by gauging and positioning the micropipette electrode 201 (that is, the FiberPort and BFT1 fiber clamp) and the back of the first fiberoptic cable 103 (for example the first fiberoptic cable 103 being a tapered fiberoptic cable). Aligning the laser by positioning one or both of the micropipette electrode 201 and the first fiberoptic cable 103 may be feasible based on the etalon effects from the stacking of the flat spectral filters. In some embodiments, the optical system 175 further includes a dichroic mirror 111 (for example, ZT488rdc, Chroma) positioned within the housing at an angle relative to the first side.

Details of the components of system 100, system 150, and/or system 175 are described as follows. In some embodiments, the second fiber optic cable 107 comprises a tapered fiber optic cable. In some embodiments, the dichroic mirror 111 is positioned at an angle of 45 degrees relative to the first side. In some embodiments, the dichroic mirror 111 is positioned at an angle of 45 degrees relative to each of the first, second, third and fourth sides. In some embodiments, the dichroic mirror 111 is planar, concave, and/or convex. In some embodiments, the dichroic mirror 111 is configured to reflect light in the range of 400 to 600 nm or about 488 nm light. In some embodiments, the dichroic mirror 111 comprises a circular dichroic mirror, which can be mounted or not mounted depending on ordering of the optical components of the system 100. In some embodiments, the dichroic mirror 111 comprises a 25 mm circular dichroic mirror. In some embodiments, the dichroic mirror 111 is angled to reflect a majority of the light from the light source 102 via the first fiber optic cable 103 and first collimator 104 to either the second collimator 106 and second fiber optic cable 107, or to the filter portion 109 and APD 108.

In some embodiments, the light source 102 comprises a laser. In some embodiments, the laser is configured to provide light in the range of 400 to 600 nm or at about 488 nm.

In some embodiments, the at least one filter 110 is configured to focus light on the APD 108. In some embodiments, the at least one filter 110 comprises five filters.

In some embodiments, the system (100, 150, 175) further includes a plurality of gaskets 112 (for example CPG3, Thorlabs) positioned between components such as, for example, the first collimator 104 and the mirror cage 101, the second collimator 106 and the mirror cage 101, and the APD 108 and the mirror cage 111. In some embodiments, the system (100, 150, 175) further includes a plurality of cage plates positioned between components such as, for example, the first collimator 104 and the mirror cage 101, and the second collimator 106 and the mirror cage 101.

In some embodiments, the APD 108 is connected to the mirror cage 101 via a bracket, a cage plate (for example, CP33T/M, Thorlabs), a 3D printed bracket, and/or the filter portion 109. In some embodiments, the filter portion 109 comprises a tube (for example, SM1 Tube, Thorlabs). In some embodiments, the filter portion 109 is connected to the mirror cage 101 via coupler (for example, SM1T3, Thorlabs). In some embodiments, the filter portion 109 includes a filter retaining ring (for example, SM1RR, Thorlabs).

In some embodiments, the first and second fiber optic cables (103, 107) comprise FC/PC, FC/APC, SMA and/or any other suitable fiber optic cables or combinations thereof. In some embodiments, the system (100, 150, 175) further includes a neutral density filter 113 (for example NE13A-A, Thorlabs) position between the energy sensor 105 and the mirror cage 101. In some embodiments, the mirror cage comprises a dichroic mirror cube (for example, CM1-DCH, Thorlabs).

In some embodiments, an automated micropipette electrode guidance system 200 includes the optical system (100, 150, 175) as described herein, and a micropipette electrode 201 connected to the optical system via the second fiber optic cable 107. In some embodiments, the system 200 is configured for simultaneous emission and collection of light from the micropipette electrode 201.

In some embodiments, a fiber port (for example, PAF2P-11C, Thorlabs) connects to the second fiber optic cable 107 via a FC/PC port such as a multimode wideband circulator (for example, WMC2L1F, Thorlabs). In some embodiments, the circulator connects to a FC/PC-FC/PC adapter (for example, ADAFC2, Thorlabs), a 250 μm ferrule FC/PC connector (for example, B30250C, Thorlabs) and a bare fiber terminator (for example, BFT1, Thorlabs). In some embodiments the system 200 terminates into a tapered fiber optic.

In some embodiments, the system 200 includes a straight near-field scanning optical microscopy (NSOM) fiber probe with a MM-UV (200-1200 nm) wavelength fiber, a 50 μm core, a 125 μm cladding diameter, a 1500 nm aperture diameter, and a Cr—Au coating. In some embodiments, no electrical contact wire and/or no electrical connector are used in the straight NSOM fiber probe. In some embodiments, the straight NSOM fiber probe has a length of about 3 m.

In some embodiments, the optical system (100, 150, 175) is configured for use as an optical unit of a micropipette or multipipette guidance system, such as for the system described in U.S. patent application Ser. No. 17/401,640 filed Aug. 13, 2021, and entitled “Fluoro-acoustic multipipette electrode and methods of use therefor”, and/or the system described in Christopher Miranda et al., “Automated microscope-independent fluorescence-guided micropipette,” Biomed. Opt. Express 12, 4689-4699 (2021), each of which is hereby incorporated herein by reference in its entirety.

In some embodiments, the micropipette can be configured for optical guidance, acoustic guidance, electrical guidance, or a combination thereof. In some embodiments, a micropipette system includes a micropipette having a hollow glass tip, and a headstage configured to detect electrical resistance measurements at the hollow glass tip. In some embodiments, the micropipette system further includes a guidance system including an actuator configured to move the hollow glass tip in one or more degrees of freedom to position the micropipette. In some embodiments, the micropipette system further includes at least one light source, such as light source 102, coupled to the recording electrode and the micropipette via an optical system, such as optical system 100, 150, or 175, configured to emit light from the hollow glass tip. In some embodiments, the micropipette system further includes an ultrasound transducer configured to detect one or more photoacoustic signals in response to the light. In some embodiments, the micropipette system further includes a light sensor, such as APD 108, configured to detect one or more optical signals in response to the light. In some embodiments, the micropipette system further includes a computing system including a processor and a non-transitory computer-readable medium for performing positioning of the micropipette via actuation of the guidance system based on received optical, electrical and/or acoustic signals. In some embodiments, the micropipette has a tapered fiber optic inside and a piece of wire that is further proximal than the tapered fiber optic. In some embodiments, the wire is Ag, or Ag/Cl.

In some embodiments, the automated micropipette electrode guidance method includes providing the optical system (100, 150, 175) as described above and providing a micropipette electrode 201 connected to the optical system (100, 150, 175) via the second fiber optic cable 107 as described above. The method can further include supplying a first portion of light to the micropipette electrode 201 via the light source 102 and optical system (100, 150, 175). The method can further include measuring a second portion of light on the energy sensor 105. The method can further include measuring collected light from the micropipette electrode 201 with the APD 108.

In some embodiments, the step of supplying a first portion of light to the micropipette electrode 201 comprises supplying light via the light source 102, the first fiber optic cable 103, the first collimator 104, the dichroic mirror 111, the second collimator 106, and the second fiber optic cable 107 to the micropipette electrode 201. In some embodiments, the step of measuring a second portion of light comprises measuring light supplied by the light source 102 that was transmitted through and not reflected off of the dichroic mirror 111.

In some embodiments, the step of measuring collected light from the micropipette electrode 201 with the APD 108 comprises measuring light received from the micropipette electrode 201 via the second fiber optic cable 107, the second collimator 106, that passes through the dichroic mirror 111, and the at least one filter 110. In some embodiments, the supplied light is light in the range of 400 to 600 nm or about 488 nm and is supplied by a laser.

### Experimental Examples

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out exemplary embodiments of the present invention, and are not to be construed as limiting in any way the remainder of the disclosure.

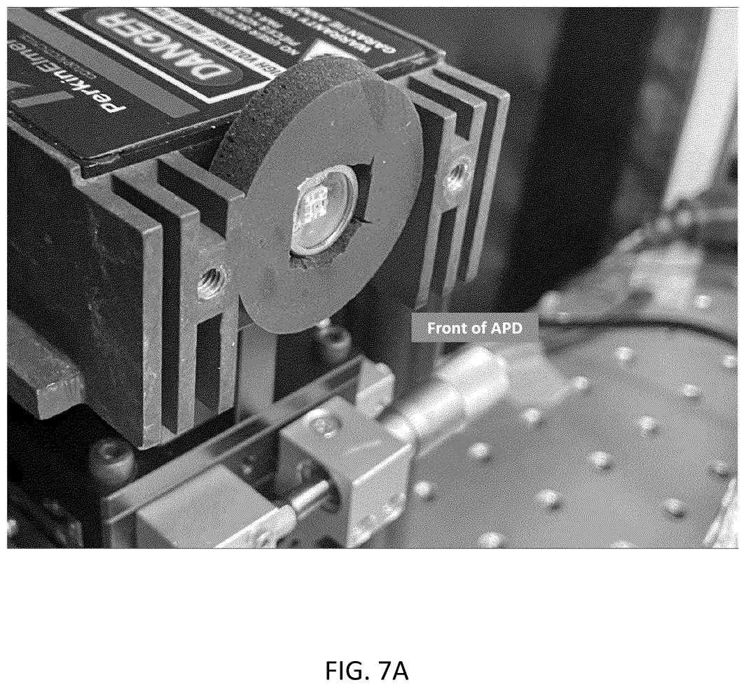

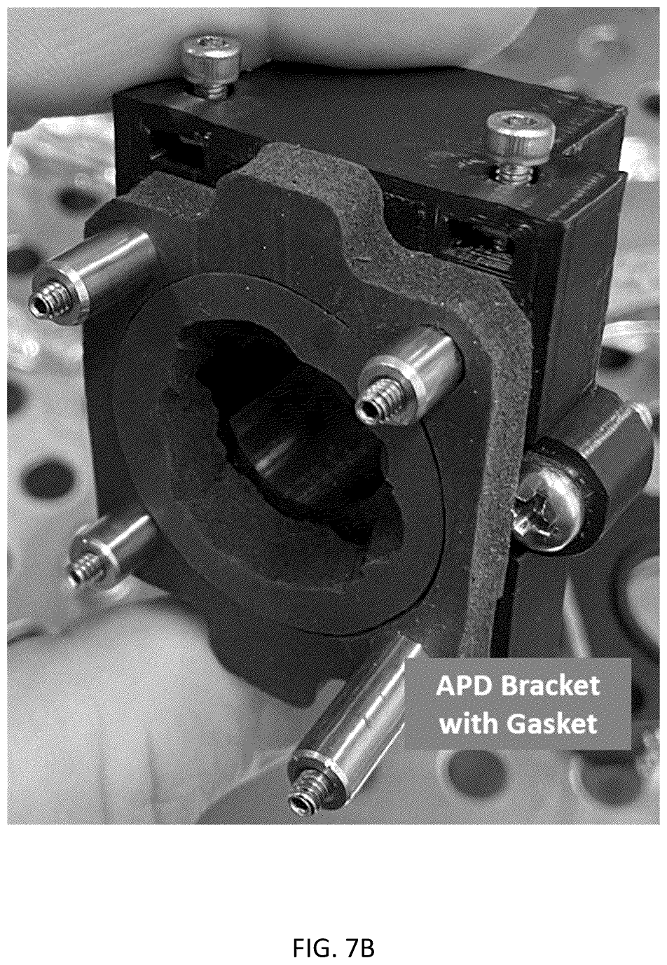

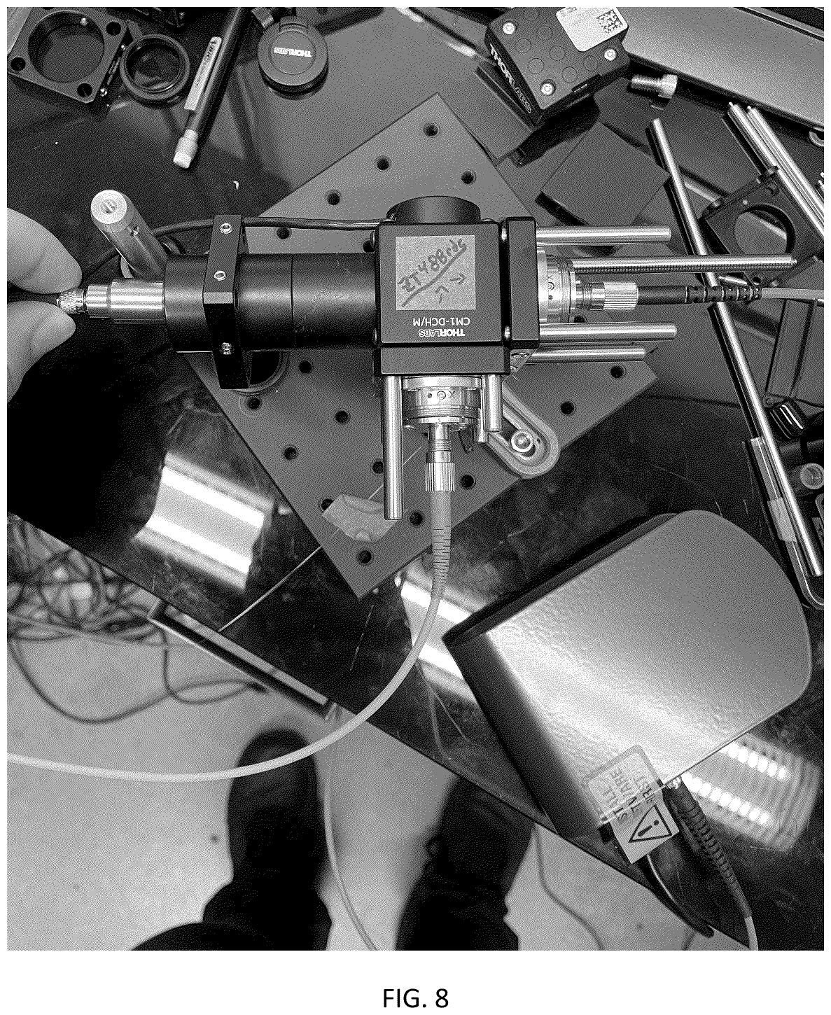

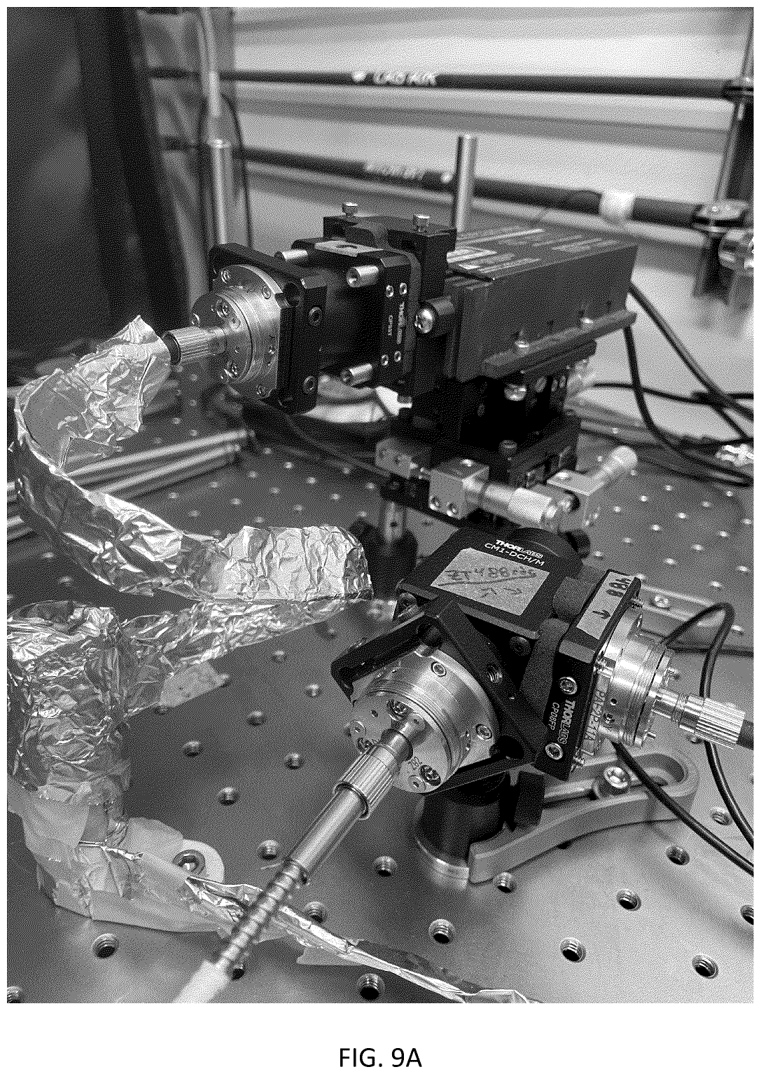

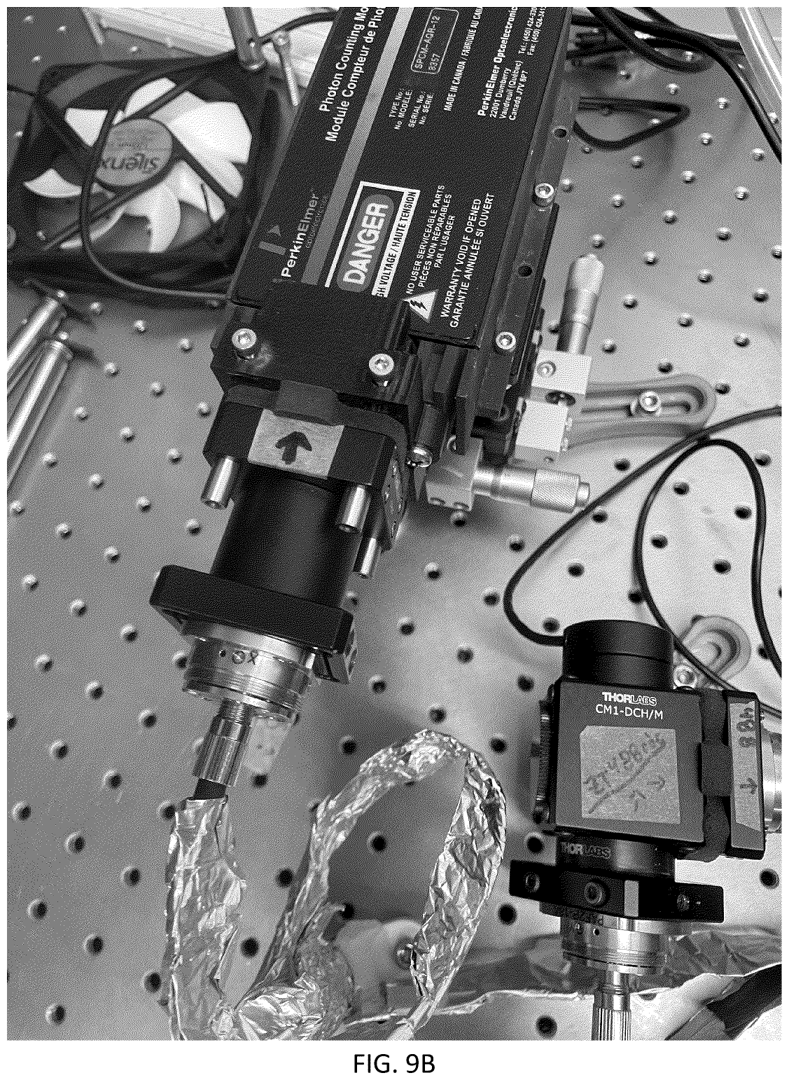

Referring now to FIGS. 7A to 7B, FIG. 8, and FIGS. 9A to 9B, exemplary experimental automated optical micropipette electrode guidance systems are shown. FIG. 7A shows the front of an APD, FIG. 7B shows a bracket and gasket configured to connect to the APD, FIG. 8 shows a top-down view of the experimental system 100, and FIGS. 9A-9B show perspective views of the system 150. The systems (100, 150) are configured to combine complete optical and electrophysiology components for 2-way optical detection from the tip of the micropipette electrode. The goal of developing this tool was to enable translation from the research and development lab to external electrophysiology labs for research applications.

In some embodiments, the systems (100, 150) allow simultaneous emission and collection of light via a tapered fiber optic integrated into a micropipette electrode. In the particular experimental examples shown, 488 nm light originates from an FC/PC port, is redirected at a 90-degree angle through a dichroic mirror (zt488rdc, Chroma) and transferred into a FiberPort collimator (PAF2P-18A, Thorlabs) before traveling into the flat end of a fiber optic. As some energy is transmitted, rather than reflected, an energy sensor (S120C, Thorlabs) is placed on the opposite end of the dichroic mirror cage cube to sample the output of the 488 nm laser. In collection mode, light gathered by the tapered fiber optic exits the flat end of the fiber, passes through the mirror, and is focused onto the sensor of the APD.

In some embodiments, the micropipettes are produced from filament-free borosilicate glass tubes. The original tubes have an outer diameter of 1.5 mm, inner diameter of 1.1 mm, and are 10 cm in length. In one example, the glass tubes are ITEM #: B150-110-10HP from Sutter Instruments. These are then “pulled” from a P-87 micropipette puller (Sutter Instruments). These capillary tubes are subjected to cycles of heat and pulling, with parameters of heat temperature, pull velocity, and length of heating time. The parameters of heat, temperature, pull velocity, and length of heating time are optimized to produce micropipettes with the ideal taper and aperture size. In some examples, 2 micropipettes are produced simultaneously from one capillary tube as the original tube separates at the halfway point producing two units. In some examples, a resistance measurement of the micropipette is used to gauge aperture size. The resistance used for gauging aperture size was 1-1.3 Mega-Ohms.

The following publications are each hereby incorporated herein by reference in their entirety:

U.S. patent application Ser. No. 17/401,640 filed Aug. 13, 2021, and entitled “Fluoro-acoustic multipipette electrode and methods of use therefor.”

Christopher Miranda, Madeleine R. Howell, Joel F. Lusk, Ethan Marschall, Jarrett Eshima, Trent Anderson, and Barbara S. Smith, “Automated microscope-independent fluorescence-guided micropipette,” Biomed. Opt. Express 12, 4689-4699 (2021)

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. An optical system, comprising:
a mirror cage;
a light source optically connected to a first side of the mirror cage via a first fiberoptic cable and a first collimator;
an energy sensor optically connected to a second side of the mirror cage opposite the first side;
a second fiber optic cable optically connected to a third side of the mirror cage via a second collimator;
an avalanche photodiode (APD) optically connected to a fourth side of the mirror cage opposite the third side; and
a dichroic mirror positioned within the housing at an angle relative to the first side.

2. The system of claim 1, wherein the second fiber optic cable comprises a tapered fiber optic cable.

3. The system of claim 1, wherein the dichroic mirror is positioned at an angle of 45 degrees relative to at least one of the first side, the second side, the third side, or the fourth side.

4. The system of claim 1, wherein the dichroic mirror is planar, concave or convex.

5. The system of claim 1, wherein the light source comprises a laser.

6. The system of claim 5, wherein the laser is configured to provide light in the range of 400 to 600 nm.

7. The system of claim 1, wherein the at least one filter is configured to focus light on the APD.

8. The system of claim 1, wherein the dichroic mirror is configured to reflect light in the range of 400 to 600 nm.

9. The system of claim 1, wherein the system further comprises a plurality of gaskets positioned between the first collimator and the mirror cage, the second collimator and the mirror cage, and the APD and the mirror cage.

10. The system of claim 1, wherein the first and second fiber optic cables comprise FC/PC fiber optic cables.

11. The system of claim 1, further comprising a neutral density filter positioned between the energy sensor and the mirror cube.

12. The system of claim 1, wherein the APD is directly connected to the fourth side of the mirror cage via a portion housing at least one filter.

13. The system of claim 1, wherein the APD is indirectly connected to the fourth side of the mirror cage via a portion housing at least one filter and a third fiber optic cable.

14. An automated micropipette electrode guidance system, comprising:
the optical system of claim 1; and
a micropipette electrode connected to the optical system via the second fiber optic cable.

15. The system of claim 16, wherein the system is configured for simultaneous emission and collection of light from the micropipette electrode.

16. An automated micropipette electrode guidance method, comprising:
providing the optical system of claim 1;
providing a micropipette electrode connected to the optical system via the second fiber optic cable;
supplying a first portion of light to the micropipette electrode via the light source and optical system;
measuring a second portion of light on the energy sensor; and
measuring collected light from the micropipette electrode with the APD.

17. The method of claim 16, wherein the step of supplying a first portion of light to the micropipette electrode comprises supplying light via the light source, the first fiber optic cable, the first collimator, the dichroic mirror, the second collimator, and the second fiber optic cable to the micropipette electrode.

18. The method of claim 16, wherein the step of measuring a second portion of light comprises measuring light supplied by the light source that was transmitted through the dichroic mirror.

19. The method of claim 16, wherein the step of measuring collected light from the micropipette electrode with the APD comprises measuring light received from the micropipette electrode via the second fiber optic cable, the second collimator, the dichroic mirror, and the at least one filter.

20. The method of claim 16, wherein the supplied light is in the range of 400 to 600 nm and is supplied by a laser.
